(19) United States
(12) Patent Application Publication
(10) Pub. No.: US 2021/0290955 A1
(43) Pub. Date: Sep. 23, 2021

(54) DEVICES, SYSTEMS, AND METHODS FOR PLANNING AND/OR CONTROL NEUROMODULATION

(71) Applicant: ONWARD MEDICAL B.V., Eindhoven (NL)

(72) Inventors: Robin BROUNS, Eindhoven (NL); Miroslav CABAN, Renens (CH)

(73) Assignee: ONWARD MEDICAL B.V., Eindhoven (NL)

(21) Appl. No.: 17/206,007

(22) Filed: Mar. 18, 2021

(30) Foreign Application Priority Data
Mar. 18, 2020 (EP) 20163794.9

(51) Int. Cl. A61N 1/36; A61N 1/372; A61B 5/11; A61B 5/00

(52) U.S. Cl. CPC A61N 1/36003; A61N 1/36067; A61N 1/37247; A61B 5/112; A61B 2560/0487; A61B 5/743; A61B 5/7435; A61B 5/4836; A61B 5/1128

(57) ABSTRACT

The present disclosure provides devices, systems, and methods for neuromodulation. In some exemplary embodiments, a neuromodulation system is provided. In some embodiments, the neuromodulation system includes a user interface device configured to display graphical information on a screen. The graphical information includes a plurality of graphical objects representing a time sequence of a plurality of stimulation patterns along a timeline, each of the plurality of stimulation patterns corresponding to at least one of a motoric function or an autonomic function. The user interface device is configured to change a shape or a position of at least one of the plurality of graphical objects along the timeline in response to a user input.

### CROSS-REFERENCE TO RELATED APPLICATION

This application claims the benefit of priority of European Patent Application No. EP20163794.9, filed Mar. 18, 2020, titled “A PLANNING AND/OR CONTROL SYSTEM FOR A NEUROMODULATION SYSTEM,” the entire disclosure of which is incorporated herein by reference.

### TECHNICAL FIELD

The present disclosure relates to neuromodulation systems, such as a neurostimulation system for a patient, and in particular, to planning and/or control systems for neuromodulation systems and methods of use.

### BACKGROUND

The spinal cord is an integral part of the central nervous system (CNS). Spinal cord injury (SCI), and also other disorders (e.g. stroke, multiple sclerosis, autonomic failure, autonomic neuropathy or cancer of the neurological tissue which impair operation of descending sympathetic pathways that normally facilitate control of autonomic functions) can result in motor deficits. For instance, SCI interrupts the communication between the spinal cord and supraspinal centers, depriving these sensorimotor circuits from the excitatory and modulatory drives necessary to produce movement. However, SCI results also in sensory deficits and in autonomic dysfunctions. For example, SCI results in disconnection of some, most, or all descending sympathetic pathways that carry signals responsible for regulating arterial blood pressure, heart rate, and/or gut and/or bladder function.

Spinal cord stimulation (SCS) is a well-established neuromodulatory therapy not only for restoring locomotion/motoric function after spinal cord injury or central nervous diseases, but also for treating inter alia pain and/or restoring autonomic function.

Neuromodulation systems have been developed for the treatment of dysfunction, damage, disease and/or disorders of the nervous system. A neuromodulation system, such as a neurostimulation system, for a patient suffering from motoric dysfunction and/or autonomic dysfunction requires programming to define which stimulation parameter settings, may be used to evoke certain muscles or muscle groups. Such muscles and/or muscle groups may be responsible for locomotion of the arms or legs, and/or responsible for, for example, bowel movement, sphincter control, bladder control and/or sexual function. Such programming of stimulation parameters may be performed by a clinical professional, a physiotherapist, and/or the patient himself, and can be facilitated by a computer-driven application.

Once stimulation parameters have been found to stimulate certain muscles (or groups of muscles), the muscular activations need to be sequenced in time. For example, walking is defined by alternating stimulations of specific muscles on the right and on the left leg. Similarly, other kinds of locomotion, e.g. running, swimming, cycling, rowing, can be thought of as a time sequence of muscular stimulations.

Simulation parameters may need to be adapted to specific needs of a patient. For example, the stimulation parameters may need to be modified based on the patient's specific pathological symptoms or the patient's progress or response to treatment. The stimulation parameters may require frequent and accurate modifications and/or precise timing of start and stop during an exercise or stimulation treatment of the patient. In some instances, it is desirable to modify the stimulation parameters in real-time to improve the result of the stimulation. Moreover, it is also desirable to allow the clinical professional, physiotherapist, and/or the patient to modify the stimulation parameters and the timing of the stimulation parameters as needed accurately in a fast, simplified and efficient manner.

US 2014/0172045 A1 generally describes a method for programming a neurostimulator. One or more control elements may be actuated to select the series of steps from a plurality of series of steps stored in a memory of an external control device. One or more control elements may be actuated during the performance of the series of steps in order to cause one of the steps to pause, stop, restart, skip, or repeat. The series of steps may be a series of pre-programming steps, and the method may further include programming the neurostimulator after the series of pre-programming steps is performed. An external device for programming the neurostimulator includes control circuitry configured for automatically performing the series of steps, and a user interface including the one or more control elements configured for being actuated. The control device also includes the memory for storing the plurality of series of steps.

EP 3328481 generally describes a neurostimulation system including a programming control circuit and a user interface. The programming control circuit may be configured to generate a plurality of stimulation parameters controlling delivery of neurostimulation pulses according to one or more neurostimulation programs each specifying a pattern of the neurostimulation pulses. The user interface includes a display screen, a user input device, and a neurostimulation program circuit. The neurostimulation program circuit may be configured to allow for construction of one or more pulse trains (PTs) and one or more train groupings (TGs) of the one or more neurostimulation programs, and to allow for scheduling of delivery of the one or more neurostimulation programs, using the display screen and the user input device. Each PT includes one or more pulse blocks each including a plurality of pulses of the neurostimulation pulses. Each TG includes one or more PTs.

None of the aforementioned methods or systems solve the above-discussed problems.

### SUMMARY

According to an exemplary embodiment of the present disclosure, a neuromodulation system is provided. In some embodiments, the neuromodulation system includes a user interface device. In some embodiments, the user interface device is configured to display graphical information on a screen. In some embodiments, the graphical information includes a plurality of graphical objects representing a time sequence of a plurality of stimulation patterns along a timeline, each of the plurality of stimulation patterns corresponding to at least one of a motoric function or an autonomic function. In some embodiments, the user interface device is configured to change a shape or a position of at least one of the plurality of graphical objects along the timeline in response to a user input. In some embodiments, the user interface device is configured to update the display of the plurality of graphical objects on the screen. In some embodiments, the user interface device is configured to generate a first updated time sequence of the stimulation patterns corresponding to the updated display of the plurality of graphical objects. In some embodiments, the neuromodulation system includes a stimulation pattern programming device. In some embodiments, the stimulation pattern programming device is configured to receive the first updated time sequence of the stimulation patterns. In some embodiments, the stimulation pattern programming device is configured to provide, to a stimulation device, instructions to stimulate, using a first plurality of electrodes, one or more anatomical structures based on the first updated time sequence of the stimulation patterns.

According to an exemplary embodiment of the present disclosure, a non-transitory computer-readable storage medium is provided. The non-transitory computer-readable storage medium stores a set of instructions that, when executed by one or more processors, cause the one or more processors to perform a method of neuromodulation. In some embodiments, the method includes displaying, by a user interface device, graphical information on a screen. In some embodiments, the graphical information includes a plurality of graphical objects representing a time sequence of a plurality of stimulation patterns along a timeline, each of the plurality of stimulation patterns corresponding to at least one of a motoric function or an autonomic function. In some embodiments, the method includes changing, by the user interface device, a shape or a position of at least one of the plurality of graphical objects along the timeline in response to a user input. In some embodiments, the method includes updating, by the user interface device, the display of the plurality of graphical objects on the screen. In some embodiments, the method includes generating, by the user interface device, a first updated time sequence of the stimulation patterns corresponding to the updated display of the plurality of graphical objects. In some embodiments, the method includes receiving, by a stimulation pattern programming device, the first updated time sequence of the stimulation patterns. In some embodiments, the method includes providing, by the stimulation pattern programming device to a stimulation device, instructions to stimulate, using a first plurality of electrodes, one or more anatomical structures based on the first updated time sequence of the stimulation patterns.

According to an exemplary embodiment of the present disclosure, a neuromodulation system is provided. In some embodiments, the neuromodulation system includes a user interface device. In some embodiments, the user interface device configured to display a patient avatar on a screen; update a position of one or more body parts of the patient avatar in response to user input; and generate a time sequence of a plurality of stimulation patterns based on the updated position of the one or more body parts. In some embodiments, the neuromodulation system includes a stimulation pattern programming device. In some embodiments, the stimulation pattern programming device is configured to receive the time sequence of the plurality of stimulation patterns; and provide, to a stimulation device, instructions to stimulate, using a plurality of electrodes, one or more anatomical structures of the patient based on the time sequence of the plurality of stimulation patterns.

Additional disclosure of the disclosed embodiments will be set forth in part in the description that follows.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain exemplary principles of certain disclosed embodiments as set forth in the accompanying claims.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 depicts a schematic representation of an exemplary embodiment of a planning and/or control system, according to some embodiments of the present disclosure.

FIG. 2 depicts a schematic representation of an exemplary embodiment of a planning and/or control system, according to some embodiments of the present disclosure.

FIG. 2A depicts a schematic representation of an exemplary embodiment of a planning and/or control system, according to some embodiments of the present disclosure.

FIG. 2B depicts a schematic representation of an exemplary embodiment of a planning and/or control system, according to some embodiments of the present disclosure.

FIG. 3 depicts a schematic representation of an exemplary embodiment of a planning and/or control system, according to some embodiments of the present disclosure.

FIG. 3A depicts a schematic representation of an exemplary embodiment of a planning and/or control system, according to some embodiments of the present disclosure.

FIG. 3B depicts a schematic representation of an exemplary embodiment of a planning and/or control system, according to some embodiments of the present disclosure.

FIG. 4 depicts exemplary graphical information provided by an exemplary embodiment of a graphical presentation module, according to some embodiments of the present disclosure.

FIG. 5 depicts exemplary graphical information provided by an exemplary embodiment of a graphical presentation module, according to some embodiments of the present disclosure.

FIG. 6 depicts exemplary graphical information provided by an exemplary embodiment of a graphical presentation module, according to some embodiments of the present disclosure.

FIG. 7 depicts exemplary graphical information provided by an exemplary embodiment of a graphical presentation module, according to some embodiments of the present disclosure.

FIG. 8 depicts exemplary user interactions with graphical information displayed on a screen to ramp up or ramp down a stimulation pattern, according to some embodiments of the present disclosure.

FIG. 9 depicts an example of a timeline embodied as a circle that repeats itself, according to some embodiments of the present disclosure.

FIG. 10 depicts an example of how acoustic information can be provided, according to some embodiments of the present disclosure.

FIG. 11 depicts a schematic representation of an exemplary embodiment for updating stimulation patterns, according to some embodiments of the present disclosure.

FIG. 12 depicts an example of superimposing camera recordings on intended locomotion of a patient, according to some embodiments of the present disclosure.

FIG. 13 depicts exemplary graphical information provided by an exemplary embodiment of a graphical presentation module, according to some embodiments of the present disclosure.

FIG. 14 depicts an exemplary of manipulation and/or modulation of movement of a patient on an exemplary avatar, according to some embodiments of the present disclosure.

### DETAILED DESCRIPTION

Reference will now be made in detail to exemplary embodiments. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the devices, systems, methods, and examples are illustrative only and are not intended to be necessarily limiting.

Disclosed embodiments provide a neuromodulation system for a neuromodulation system, such as a neurostimulation system for a patient that enables optimal sequence of responses to stimulation, e.g. on smooth movements of a patient, by facilitating time sequencing of one or more stimulation patterns. A stimulation pattern includes one or more stimulation parameters, including, but not limited to, an amplitude, frequency, and pulse width. Although some embodiments are described with respect to one or more stimulation patterns and time sequencing of one or more stimulation patterns, disclosed embodiments equally apply to one or more stimulation parameters and time sequencing of one or more stimulation parameters.

In some embodiments, a neuromodulation system, such as a neurostimulation system, includes a planning and/or control system. In some embodiments, the planning and/or control system is operatively connected to and communicates with a stimulation device of the neuromodulation system. In some embodiments, the stimulation device stimulates, using one or more electrodes, one or more anatomical structures of the patient. In some embodiments, a planning and/or control system for a neuromodulation system includes a stimulation pattern programming module configured and arranged for receiving and processing data on stimulation patterns or stimulation parameters and a time sequence of the stimulation patterns or stimulation parameters, and a graphical presentation module configured and arranged for providing graphical information about the stimulation patterns or stimulation parameters and the time sequence of the stimulation patterns or stimulation parameters. In some embodiments, the stimulation pattern programming module is a stimulation pattern programming device. In some embodiments, the graphical presentation module is a device comprising a user interface. In some embodiments, the user interface includes a screen. In some embodiments, the screen is a touchscreen.

The present disclosure provides, in the context of neuromodulation, such as neurostimulation, stimulation patterns or stimulation parameters that can be adapted to specific needs of a patient. In some instances, stimulation patterns or stimulation parameters may be patient specific and/or require frequent modifications and/or precise timing of start and stop. In some instances, some patients may move faster/slower (such as walking) than others, requiring neurostimulation to succeed in a respective faster or slower fashion. Furthermore, the support required from stimulation may vary on the patient's specific pathological symptoms. Thus, the stimulation sequence may involve different muscles or muscle groups and they may be required at different moments or for different durations during the exercise or stimulation program. On some days, a patient may require longer (or shorter) stimulation of a certain muscle than on other days. Also, patients may be expected to progress and show improvements, placing ever new requirements on their stimulation patterns. Consistent with disclosed embodiments, a neuromodulation system, such as a neurostimulation system, for a patient, including or using a stimulation pattern programming module and a graphical presentation module enables fast, simplified, and efficient planning of a precise neuromodulation of a patient, in particular in that the outcome of the stimulation is close to the respective physiological function of a healthy and/or uninjured individual and/or patient specific.

In some embodiments, a neuromodulation system, such as a neurostimulation system includes at least one of a display, a controller, a programmer, a communication module, a telemetry module, a stimulation device, an electrode, a sensor and/or a sensor network.

In some embodiments, the stimulation pattern programming module and the graphical presentation module are part of a computing device. Non-limiting examples of the computing device include a mobile device (e.g., a smart phone, a tablet, or the like), a smart device (e.g., a smart wearable device, a smart display, or the like), or other like electronic device that can receive data from and present information to a user. For example, the stimulation pattern programming module may be or may include an interface module configured and arranged for receiving user-based stimulation parameters and/or a user-based time sequence of the stimulation parameters.

In some embodiments, the interface module may enable a user, e.g. a therapist, a physiotherapist, a physician, a trainer, a medical professional and/or a patient, to directly provide modification of the stimulation parameters (thus allowing for patient-specific modification, for example through manual sequencing of stimulation patterns). In some embodiments, the interface module may be or may include a graphical user interface, a mouse, a trackball, and/or a joystick. In some embodiments, the graphical user interface may include a display, such as a touch screen, and/or a touch pad. In some embodiments, the interface module may be configured and arranged for allowing a user to actuate at least one control element.

A planning and/or control system consistent with the disclosed embodiments may be easy for the user to use, potentially eliminating the need for time-consuming user training. In some instances, the user may manually place one or more blocks on a timeline for defining stimulation parameters, move the blocks along the timeline, and increase or decrease their durations. In some embodiments, the planning and/or control system is implemented on a computer system, such as a computing device. In some embodiments, templates may be provided for certain exercises or stimulation programs (e.g. for restoring autonomic function) and for patients with specific residual motor-functions and/or autonomic functions. In some embodiments, a template includes a plurality of blocks representing a time sequence of a plurality of stimulation patterns along a timeline. In some embodiments, the plurality of blocks is displayed along the timeline: in a plurality of rows.

In some embodiments, the user may manually change stimulation parameters and/or the time sequence of stimulation parameters and/or the blocks on the timeline by acting on control elements configured for such user actuation. Such control elements can include, but are not limited to: anchors, axes, points, knots, buttons, arrows, hand signals, emojis, crosses, windows, text, and/or shortcuts.

In some embodiments, the user may manually change stimulation parameters and/or the time sequence of stimulation parameters and/or the blocks on the timeline by acting on an avatar configured and arranged for such user actuation.

In some embodiments, the stimulation parameters and/or the time sequence of the stimulation parameters can be modulated by modifying the graphical information about the stimulation parameters and the time sequence of the stimulation parameters. Advantageously, this may enable rapid modification of stimulation parameters and subsequent stimulation of a patient, enabling rapid therapy progress, with a therapy specifically adapted to the patient's needs.

In some embodiments, the stimulation parameters and/or the time sequence of the stimulation parameters may be modulated in real-time and/or close to real-time. For example, the stimulation parameters and/or the time sequence of the stimulation parameters may be modulated during stimulation and/or task execution.

Alternatively, or additionally, the stimulation parameters and/or the time sequence of the stimulation parameters may be modulated before stimulation is provided (pre-programming of stimulation parameters and/or the time sequence of the stimulation parameters).

In some embodiments, the stimulation pattern programming module may be or may include a computer-assisted module configured and arranged for updating stimulation parameters and/or the time sequence of stimulation parameters based on feedback information provided by at least one external module and/or a set of instructions. Such feedback information and/or instructions may fine-tune the start and/or stop and/or ramping up and/or ramping down and/or repetitions, etc. of stimulation patterns or control the position of a muscle and/or stimulation block. As used herein, a stimulation block may also be referred to as muscle group.

In some embodiments, a computer-assisted module is a software module. In some embodiments, a computer-assisted module is a hardware module. In some embodiments, a computer-assisted module combines software and hardware for allowing a specialized computing device to perform the functions consistent with the disclosed embodiments. In some embodiments, the computer-assisted module may be connected to at least one external module. The at least one external module may be or may include at least one sensor and/or sensor network providing feedback information of the patient to the stimulation pattern programming module. The feedback information can include, but is not limited to: a position, motion, environment, and/or physiology/pathophysiology of the patient.

In some embodiments, at least one sensor and/or sensor network may be or may include at least one of an inertial measurement unit (IMU), an optical sensor, a camera, a piezo element, a velocity sensor, an accelerometer, a magnetic sensor, a torque sensor, a pressure sensor, a displacement sensor, a contact sensor, an EMG sensor, a goniometer, a hall sensor, a gyroscope, a motion tracking video camera, or an infra-red camera.

In some embodiments, the computer-assisted module includes a non-transitory computer-readable storage medium and one or more processors. In some embodiments, the computer-assisted module may be configured and arranged for providing at least one set of instructions, e.g. at least one algorithm. The at least one set of instructions, when executed by one or more processors, may automatically alter and/or update stimulation parameters and/or time sequencing of stimulation parameters. This may have the advantage that the stimulation parameters and/or time sequence of stimulation parameters are optimized during an ongoing motion and/or behavior of a patient, using data derived from the patient himself and/or his environment and/or a set of instructions, thus supporting efficient and rapid rehabilitation.

In some embodiments, the feedback information provided by the at least one external module and/or the at least one set of instructions may be used by the planning and/or control system, e.g. by the computer-assisted module and/or another processing module, to alter the stimulation parameters and/or time sequencing of stimulation parameters, alternatively or in addition to manually setting stimulation parameters and/or time sequencing of stimulation parameters by a user.

In some embodiments, at least one external module may be used to provide information, such as feedback information, that may be communicated to the user, e.g. acoustically and/or visually, based on which the user may program or modify the stimulation parameters and/or time sequencing of stimulation parameters, with or without automatic updates. For instance, a live EMG trace may be displayed in the background of stimulation parameters and/or time sequencing of stimulation parameters, or the video of the patient may be displayed behind an avatar, or auditory tones or spoken messages may be provided that inform the user (e.g., therapist and/or patient) of stimulation events. Such information may allow a user to update the stimulation parameters and/or time sequencing of stimulation parameters. In other words, the feedback information may aid the user to perform the programming.

In some embodiments, the computer-assisted module may update the stimulation parameters and/or the time sequence of stimulation parameters in real-time or close to real-time (e.g., with minimum delay).

In some embodiments, the planning and/or control system may be a closed-loop system or an open-loop system. In some embodiments, the planning and/or control system allows both closed-loop or open-loop functionality. In this regard, the user may switch between these options or there may be routines or control elements that can do or propose such a switch from closed-loop to open-loop and vice versa.

In some embodiments, the planning and/or control system may be used online and/or offline. In some embodiments, the graphical presentation module may be configured and arranged for providing graphical information about the time sequence of the stimulation parameters. In some embodiments, the graphical information about the time sequence of the stimulation parameters is provided as a timeline, a graph, a plot, a table, a circular pattern and/or a clock. Thus, the time sequence of the stimulation parameters is presented to the user in a graphically appealing and easy layout, which can be easily understood by the user. This has the advantage that, due to the simplicity of the operation and the layout, the number of errors that occur when operating is reduced.

In some embodiments, to provide the user a better guidance for improving the patient's stimulation parameters, image data can be combined with reference outcome patterns, such as optimal outcome patterns. In various embodiments, the external module can include a camera that provides camera recordings and/or frames of the patient. In some embodiments, such camera recordings and/or frames could be superimposed onto reference outcome patterns. Such reference outcome patterns could be locomotion or movements of a healthy subject. As such, the user could see which muscles need more and/or less activation to match a reference locomotion or movement, e.g. an optimal walking pattern. This could be applied to various kinds of locomotion activity and/or motoric function. In some embodiments, physiological measurements by one or more sensors may be added and/or superimposed to stimulation parameters.

In some embodiments, one or more sensors may be applied on a patient to measure when muscles are activated voluntarily by the patient. The sensor signals could be displayed in the same time sequence of stimulation parameters. In general, feedback information provided by the sensors may be used to better define stimulation parameters and to align with the patient's voluntary activity. For example, one or more sensors can be used to capture the motion or muscular activity of the patient. The sensor signals can be compared against one or more optimal signals, such as sensor signals of a healthy patient. Deviations of the sensor signals from the one or more optimal signals, in space and/or time, can be corrected by adjusting one or more stimulation patterns and/or the time sequence of the stimulation patterns.

In some embodiments, timings of start and stop of stimulation of a certain muscle needs to be accurate. For instance, during locomotion, muscular action succeeds in rapid fashion and muscles may need to be timed to each other sometimes down to 10 ms to 80 ms, for example. Providing graphical information about the time sequence of stimulation parameters, e.g. as a timeline, allows the user to judge the patient's movements visually. As a non-limiting example, as for locomotion, when many muscles and/or stimulation blocks and/or stimulation parameters are involved, it may be challenging for a user to bring everything together and produce a therapeutic, effective timeline of stimulation parameters. In some embodiments, providing graphical information of the time sequence of stimulation parameters, as a timeline, a graph, a plot, a table, a circular pattern, a clock may enable the user to bring everything together and produce a therapeutic effective time sequence of stimulation parameters. Advantageously, rapid and correct modification of stimulation parameters and subsequent stimulation of a patient, enabling rapid therapy progress, with a therapy specifically adapted to the patient's needs can therefore be enabled.

Additionally, or alternatively, a combination of different layouts may be possible, provided as graphical information in black and white and/or color.

In some embodiments, stimulation parameters may include amplitude, frequency, and/or pulse width of the stimulation of at least one muscle, a muscle group, at least one stimulation block, and/or at least one anatomical structure. In some embodiments, the time sequence of a stimulation pattern may include a start time, a stop time, an up ramping, a down ramping, a duration, repetition and/or cycles of stimulation of at least one muscle, a muscle group, at least one stimulation block, and/or at least one anatomical structure. Ramping up and/or ramping down may be important to facilitate gradual transitioning of stimulation, rather than an immediate violent application of stimulation that the patient overcome with. Disclosed embodiments enable the stimulation parameters required for a complex process, such as walking, standing up, sitting down, cycling, swimming, grasping, blood pressure control, bladder control, etc., to be accurately and efficiently defined, finally enabling smooth movements and/or defined physiological functions.

In some embodiments, a time sequence of stimulation patterns or stimulation parameters may be repeated and/or cycled to allow for performance of sustained locomotion, e.g. walking, running, cycling, swimming and/or rowing. In sustained locomotion, the timeline of stimulation patterns or stimulation parameters may automatically replay in the time sequence of stimulation patterns or stimulation parameters. In some embodiments, stimulation programs for autonomic functions may be created using such repeated or cyclic timelines. In some embodiments, a stimulation program can include a time sequence of stimulation patterns or stimulation parameters.

In some embodiments, the time sequence of stimulation parameters runs once and then stimulation is stopped. In some embodiments, the time sequence of stimulation parameters runs once and then keeps applying the last stimulation pattern of the time sequence, for example, indefinitely until manually stopped by a user or by a sensor that detects patient-voluntary initiation of another type of locomotion. As a non-limiting example, following application of a “standing up” template, a patient may require continuing stimulation to remain standing upright (e.g., such stimulation may be provided indefinitely until manually stopped by a user).

In some embodiments, the planning and/or control system may further include an acoustic module being configured and arranged for providing acoustic information about the stimulation parameters and/or the time sequence of the stimulation parameters. This has the advantage that a user may focus visually on the patient's movements and simply rely on auditory cues to understand a stimulation provided by a neuromodulation system. Focusing on the patient may allow the user to more quickly intervene to guarantee the patient's safety. Additionally, acoustic information may allow the patient to modulate his or her self-invoked movements to align with stimulations provided, to the benefit of therapy. In other words, this may aid the user to perform the programming. In some embodiments, the acoustic module includes an audio device operatively connected to the stimulation pattern programming module and/or the graphical presentation module.

In some embodiments, a system or a device may play an auditory cue/tone as soon as stimulation of a stimulation block and/or a muscle is initiated and/or stopped. As described herein, the system or a device that produces the auditory cue may not be limited to the system providing the means for creation of the stimulation pattern.

In some embodiments, the auditory cue may be a simple tone and/or a single frequency, multiple frequencies, or may even be a vocal cue, such as a voice reading out the stimulation parameters. The vocals may also provide a count-down before stimulation occurs. This may help the patient to align and prepare his self-invoked movements to align with these stimulations (thus increasing the efficiency of the therapy).

Consistent with disclosed embodiments, a method for performing neuromodulation is disclosed. In some embodiments, the method includes one or more of the following steps: receiving and processing one or more stimulation patterns and a time sequence of the stimulation patterns; and providing graphical information about the stimulation patterns and the time sequence of the stimulation patterns.

In some embodiments, the method further includes receiving user-based stimulation parameters and/or a user-based time sequence of the stimulation parameters. In some embodiments, the method further includes modulating the stimulation parameters and/or the time sequence of the stimulation parameters in accordance with modulation of the graphical information about the stimulation parameters and the time sequence of the stimulation parameters. In some embodiments, the method further includes updating stimulation parameters and/or the time sequence of stimulation parameters based on feedback information and/or a set of instructions. In some embodiments, the method further includes providing acoustic information about the stimulation parameters and/or the time sequence of the stimulation parameters.

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

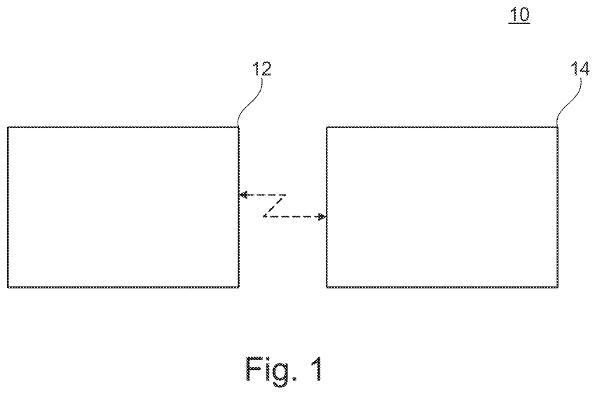

FIG. 1 depicts a schematic representation of an exemplary embodiment of a planning and/or control system 10, according to some embodiments of the present disclosure. In some embodiments, planning and/or control system 10 is part of a neuromodulation system, such as a neurostimulation system or a neurostimulation device for a patient. In some embodiments, planning and/or control system 10 is a controller external to a neuromodulation system and communicates with the neurostimulation system or neurostimulation device.

In some embodiments, system 10 includes a stimulation pattern programming module 12. In some embodiments, stimulation pattern programming module 12 is configured and arranged for receiving and processing data on stimulation patterns and a time sequence of stimulation patterns. In some embodiments, stimulation pattern programming module 12 is configured and arranged for receiving and processing data stimulation parameters and a time sequence of stimulation parameters. For example, the stimulation patterns and time sequence of the stimulation patterns can be compiled into a stimulation table that is sent to and executed by a neurostimulator or a stimulation device, or an external controller that communicates with the neurostimulator or the stimulation device. In some embodiments, for each stimulation pattern of a specific time period, the stimulation table contains a plurality of lines or columns that include the active electrodes (stimulation channels), amplitudes, frequencies, and time offsets of stimulation active during that time period.

In some embodiments, system 10 includes a graphical presentation module 14. In some embodiments, the graphical presentation module 14 is configured and arranged for providing graphical information about one or more stimulation patterns and a time sequence of the stimulation patterns. In some embodiments, the graphical presentation module 14 is configured and arranged for providing graphical information about one or more stimulation parameters and the time sequence of the stimulation parameters. In some embodiments, the graphical presentation module 14 can provide graphical information about one or more stimulation patterns and a time sequence of stimulation patterns received and processed by the stimulation pattern programming module 12. In some embodiments, the graphical presentation module 14 can provide graphical information about one or more stimulation parameters and a time sequence of stimulation parameters received and processed by the stimulation pattern programming module 12.

In some embodiments, graphical presentation module 14 includes or is a user interface device. Non-limiting examples of the user interface device include a mobile device (e.g., a smart phone, a tablet, or the like), a smart device (e.g., a smart wearable device, a smart display, or the like), or other like electronic device that can collect data and present information using a user interface. In some embodiments, the user interface device includes a display and/or one or more input/output devices such as, for example, a touchscreen, a keyboard, a mouse, a track pad, a trackball, a joystick, and the like. In some embodiments, the user interface device includes at least one storage device storage device for storing graphical information and/or stimulation patterns and time sequences of stimulation patterns, such as a memory or a physical storage device (e.g., hard drive, solid-state drive, etc.). The memory may be a random-access memory (RAM) or a read-only memory (ROM). In some embodiments, the storage device is configured to store one or more graphical templates of a time sequence of stimulation patterns predefined for restoring a locomotion/motoric function or autonomic function.

In some embodiments, the stimulation pattern programming module 12 and the graphical presentation module 14 are operatively connected. For example, as shown in FIG. 1, the stimulation pattern programming module 12 and the graphical presentation module 14 are connected by a wireless link. In some embodiments, the stimulation pattern programming module 12 and the graphical presentation module 14 are connected by one or more wires or cables. In some embodiments, the connection or communication between the stimulation pattern programming module 12 and the graphical presentation module 14 is bidirectional. In some embodiments, the connection or communication between the stimulation pattern programming module 12 and the graphical presentation module 14 is unidirectional.

Consistent with disclosed embodiments, the one or more stimulation parameters (about which graphical presentation module 14 provides graphical information) include, but are not limited to, an amplitude, frequency, and/or pulse width of the stimulation of at least one muscle, at least one stimulation block, and/or at least one anatomical structure. Consistent with disclosed embodiments, the time sequence of a stimulation pattern or a group of stimulation parameters of a stimulation pattern can include, but is not limited to, a start time, a stop time, an up ramping, a down ramping, a duration, repetition and/or cycles of stimulation of at least one muscle, at least one stimulation block, and/or at least one anatomical structure.

Consistent with disclosed embodiments, the graphical presentation module 14 can provide graphical information about the time sequence of one or more stimulation patterns or stimulation parameters in the form of a timeline T, a graph, a plot, a table, a circular pattern, and/or a clock.

In some embodiments, a neuromodulation system is provided. In some embodiments, the neuromodulation system is a neurostimulation system. In some embodiments, the neuromodulation system includes at least one of a display, a controller or a processor, a programmer, a communication module, a telemetry module, a stimulation device, an electrode, a sensor and/or a sensor network.

In some embodiments, stimulation device can be operatively connected to an Implantable Pulse Generator (IPG). The IPG can receive stimulation patterns or stimulation parameters from the controller, processor, programmer, and/or stimulation pattern programming module 12. The IPG can be configurable to simultaneously deliver independent current or voltage pulses to one or more multiple electrode arrays. The electrode arrays can be operatively connected to one or more multi-electrode arrays. These multi-electrode arrays can be designed and adapted for implantation at a location covering at least one muscle, at least one stimulation block, and/or at least one anatomical structure, such as at least a portion of the spinal cord of a subject. The IPG can apply a selective spatiotemporal stimulation of the spinal circuits and/or dorsal roots. The stimulation can be multipolar stimulation. Such system can allow effective control of locomotor functions or autonomic functions in a subject in need thereof by stimulating the spinal cord, in particular the dorsal roots, with spatiotemporal selectivity.

In some embodiments, system 10 may be used to perform a method for planning, controlling, modifying, and/or updating neuromodulation, such as neurostimulation for a patient. In some embodiments, the method includes one or more of the following steps: receiving and processing stimulation patterns and a time sequence of the stimulation patterns; and providing graphical information about the stimulation patterns and the time sequence of the stimulation patterns.

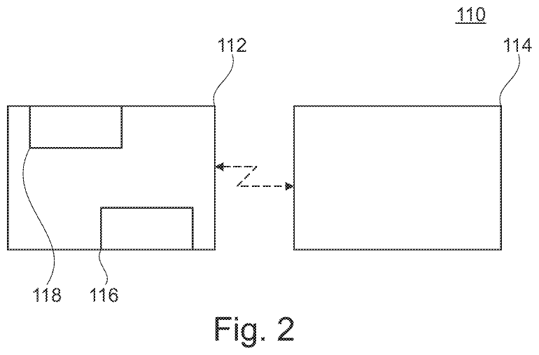

FIG. 2 depicts a schematic representation of an exemplary embodiment of a planning and/or control system 110. System 110 includes one or more of the structural and functional features as disclosed for system 10 above as shown in FIG. 1. The corresponding reference numbers in FIG. 2 for identical or similar elements or features correspond to the corresponding reference numbers of FIG. 1, and to reflect this, then this reference number is taken and increased by the value 100.

In some embodiments, as shown in FIG. 2, system 110 includes a stimulation pattern programming module 112 and a graphical presentation module 114. In some embodiments, stimulation pattern programming module 112 includes an interface module 116. In some embodiments, interface module 116 includes a communication interface, such as a wireless communication interface (e.g., a network interface) or a wired connection interface. In some embodiments, stimulation pattern programming module 112 may be an interface module 116. In some embodiments, interface module 116 is operatively connected to stimulation pattern programming module 112. In some embodiments, as shown in FIG. 2A, interface module 116 is connected to stimulation pattern programming module 112 by a wireless link, enabling bidirectional connection. In some embodiments, interface module 116 is connected to stimulation pattern programming module 112 by one or more cables, allowing for bidirectional or unidirectional connection. In some embodiments, interface module 116 is configured and arranged for receiving user-based stimulation patterns and/or a user-based time sequence of the stimulation patterns. In some embodiments, interface module 116 is configured to send one or more stimulation patterns and/or a time sequence of the stimulation patterns to a stimulation device.

In some embodiments, stimulation pattern programming module 112 includes a computer-assisted module 118. In some embodiments, computer-assisted module 118 includes a non-transitory computer-readable storage medium storing at least one set of instructions and one or more processors. The at least one set of instructions, when executed by one or more processors, may modify and/or update one or more stimulation patterns and a time sequence of the one or more stimulation patterns. In some embodiments, computer-assisted module 118 is configured and arranged for storing, modifying, and/or updating stimulation parameters and/or the time sequence of stimulation parameters based on feedback information.

In some embodiments, stimulation pattern programming module 112 includes at least one storage device storage device for storing stimulation patterns and time sequences of stimulation patterns, such as a memory or a physical storage device (e.g., hard drive, solid-state drive, etc.). The memory may be a random-access memory (RAM) or a read-only memory (ROM). In some embodiments, the storage device is configured to store one or more templates of a time sequence of stimulation patterns predefined for restoring a locomotion/motoric function or autonomic function.

In some embodiments, the stimulation pattern programming module 112 may be a computer-assisted module 118. In some embodiments, computer-assisted module 118 is operatively connected to stimulation pattern programming module 112. In some embodiments, as shown in FIG. 2B, computer-assisted module 118 is connected to stimulation pattern programming module 112 by a wireless link, enabling bidirectional connection. In some embodiments, computer-assisted module 118 is connected to stimulation pattern programming module 112 by one or more cables, allowing for bidirectional or unidirectional connection.

In some embodiments, interface module 116 and computer-assisted module 118 are also connected. In some embodiments, the stimulation parameters and/or the time sequence of the stimulation parameters are modulated dependent on modulation of the graphical information about the stimulation parameters and the time sequence of the stimulation parameters.

In some embodiments, a system (e.g., system 10, system 110, or the like) could alternatively include either an interface module 116 or a computer-assisted module 118. The interface module 116 can receive user-based stimulation parameters and/or a user-based time sequence of the stimulation parameters. The stimulation parameters and/or the time sequence of the stimulation parameters can be modulated dependent on modulation of the graphical information about the stimulation parameters and the time sequence of the stimulation parameters.

In some embodiments, system 110 can be used to perform a method for planning and/or controlling neuromodulation, such as neurostimulation, for a patient. In some embodiments, the method includes one or more of the following steps: receiving and processing stimulation patterns and a time sequence of the stimulation patterns; and providing graphical information about the stimulation patterns and the time sequence of the stimulation patterns. In some embodiments, the method includes a step of modulating the stimulation patterns and/or the time sequence of the stimulation patterns by modulation of the graphical information about the stimulation patterns and the time sequence of the stimulation patterns.

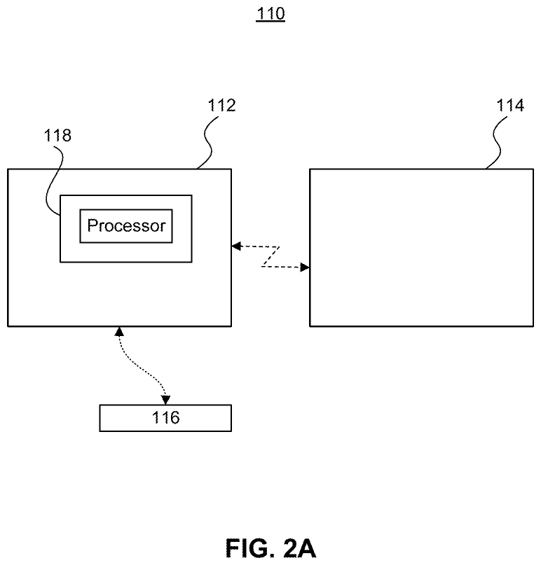

FIG. 3 depicts a schematic representation of an exemplary embodiment of a planning and/or control system 210, according to some embodiments of the present disclosure. System 210 includes the structural and functional features as disclosed for the planning and/or control system 110 disclosed with reference to FIG. 2. The corresponding reference numbers in FIG. 3 for identical or similar elements or features correspond to the corresponding reference numbers of FIG. 2 and to reflect this, then this reference number is taken and increased by the value 100.

In some embodiments, as shown in FIG. 3, system 210 is connected to an external module 220. In some embodiments, the computer-assisted module 218 is connected to the external module 220. In some embodiments, the computer-assisted module 218 is connected to the external module 220 via a bidirectional or unidirectional connection. In some embodiments, as shown in FIG. 3, computer-assisted module 218 and the external module 220 are connected by a wireless link. In some embodiments, computer-assisted module 218 and the external module 220 are connected by one or more cables or wires. In some embodiments, system 210 could be connected to more than one external module 220.

In some embodiments, as shown in FIG. 3A, external module 220 includes a sensor and/or a sensor network. In some embodiments, the sensor and/or sensor network includes at least one of an inertial measurement unit (IMU), an optical sensor, a camera, a piezo element, a velocity sensor, an accelerometer, a magnetic sensor, a torque sensor, a pressure sensor, a displacement sensor, a contact sensor, an EMG sensor, a goniometer, a hall sensor, a gyroscope, a motion tracking video camera, or an infra-red camera.

In some embodiments, external module 220 includes at least one sensor that measures motion of a patient. The at least one sensor provides feedback information of a patient equipped with the sensor. In some embodiments, the feedback information comprising at least one of a position, motion, or physiological measurement of the patient. In some embodiments, stimulation pattern programming module 212 receive feedback information from at least one sensor. In some embodiments, stimulation pattern programming module 212, using computer-assisted module 218, for example, updates stimulation patterns and/or the time sequence of the stimulation patterns based on at least in part on the feedback information. In some embodiments, stimulation pattern programming module 212 provides, to a stimulation device, instructions to stimulate, at least one muscle, at least one stimulation block. and/or at least one anatomical structure based on the updated stimulation patterns and/or the updated time sequence of the stimulation patterns. In some embodiments, the stimulation patterns and/or the time sequence of stimulation patterns are updated in real-time. In some embodiments, the stimulation patterns and/or the time sequence of stimulation patterns are updated close to real-time, with minimum delay. Additionally, or alternatively, stimulation pattern programming module 212, using computer-assisted module 218, for example, updates the stimulation patterns and/or the time sequence of stimulation patterns based on a set of instructions. In some embodiments, the set of instructions can include at least one algorithm.

FIG. 3B depicts a schematic representation of an exemplary embodiment of a planning and/or control system, according to some embodiments of the present disclosure. As shown in FIG. 3B, in some embodiments, system 210 includes an acoustic module 230. In some embodiments, the acoustic module 230 is connected to the stimulation pattern programming module 212 and the graphical presentation module 214 via a wireless link or one or more cables. In some embodiments, the connection between the acoustic module 230 and the stimulation pattern programming module 212 and/or the graphical presentation module 214 is a bidirectional connection. In some embodiments, the connection between the acoustic module 230 and the stimulation pattern programming module 212 and/or the graphical presentation module 214 is a unidirectional connection. In some embodiments, the acoustic module 230 provides acoustic information about the stimulation parameters and/or the time sequence of the stimulation parameters. In some embodiments, acoustic module 230 provides acoustic information to a user or a patient concerning a start of at least one stimulation pattern or the time sequence of a plurality of stimulation patterns.

In some embodiments, system 210 is used to perform a method for planning, controlling, modifying, and/or updating neuromodulation, such as neurostimulation, for a patient. In some embodiments, the method includes one or more of the following steps: receiving and processing stimulation patterns and a time sequence of the stimulation patterns; and providing graphical information about the stimulation patterns and the time sequence of the stimulation patterns. In some embodiments, the method includes a step of modulating stimulation patterns and the time sequence of the stimulation patterns by modulation of the graphical information about the stimulation patterns and the time sequence of the stimulation patterns. In some embodiments, the method includes a step of updating stimulation patterns and the time sequence of the stimulation patterns based on feedback information and/or a set of instructions. In some embodiments, the method includes a step of providing acoustic information about the stimulation patterns and the time sequence of the stimulation patterns.

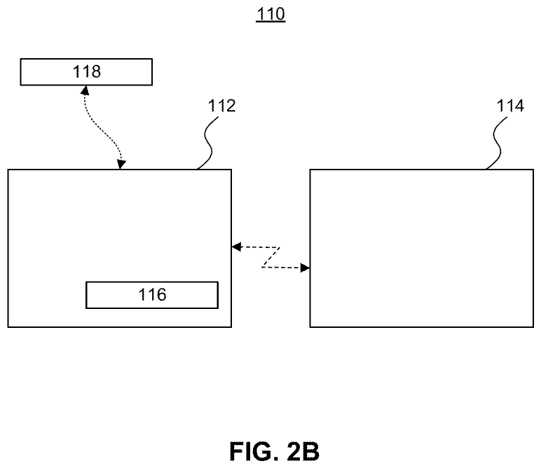

In some embodiments, graphical information includes one or more graphical objects representing a time sequence of one or more stimulation patterns along a timeline. In some embodiments, the one or more stimulation patterns corresponds to at least one of a motoric function or an autonomic function. FIG. 4 depicts exemplary graphical information provided by an exemplary embodiment of a graphical presentation module 14, 114, 214, according to some embodiments of the present disclosure. In some embodiments, graphical presentation module 14, 114, 214 displays the graphical information on a screen.

FIG. 4 shows an exemplary timeline T for defining a walking exercise for a patient. FIG. 4 shows a generic abstract illustration of a time sequence of a plurality of graphical objects, for example, blocks B, along the timeline T arranged in multiple rows. In some embodiments, a block B represents a stimulation pattern for stimulating at least one muscle, at least one stimulation block, and/or at least one anatomical structure corresponding to at least one of a motoric function or an autonomic function. In some embodiments, a block B represents a stimulation pattern for sending to one or more electrodes designed and adapted for implantation at one or more locations of the spinal cord corresponding to at least one of a motoric function or an autonomic function. In some embodiments, a stimulation pattern represented by block B includes one or more stimulation parameters.

The exemplary timeline T features stimulation patterns for stimulating certain muscle groups. For example, the exemplary timeline T as shown in FIG. 4 features stimulation patterns for the muscle groups: hip muscles, knee muscles, and ankle muscles. In some embodiments, stimulation patterns for every muscle, muscle group, stimulation block, and/or muscle fiber could be shown. In some embodiments, the timeline T features stimulation patterns for certain muscle(s), such as rectus femoris. In some embodiments, the timeline T features stimulation patterns for certain stimulation blocks. In some embodiments, the stimulation patterns refer to a collection of muscles (1 or more) that are agonistic/synergistic to achieve a certain movement. For example, the movement is bending the hip. In this example, the stimulation patterns for hip muscles, knee muscles, and ankle muscles are each set on the timeline T as a block B. The stimulation pattern for hip muscles is set on the timeline T as a block B to last from 0 seconds to 0.85 seconds, thereby lasting for 0.85 seconds. Consistent with disclosed embodiments, a muscle-group could equally mean a synergistic muscle activation (e.g., withdrawal reflex), which is not necessarily focused on an isolated joint movement.

In some embodiments, one or more graphical objects, such as blocks B, can be created, modified, and/or moved. Although some embodiments are described with respect to block B, disclosed embodiments equally apply to other types or shapes of graphical objects.

In some embodiments, in response to a user input, graphical presentation module 14, 114, 214 changes a shape or a position of at least one block B along the timeline T. In some embodiments, changing the shape or the position of at least one block B includes changing at least one of a width, location, or edge of the at least one block B. In some embodiments, in response to a user input, graphical presentation module 14, 114, 214 changes the position of a block B to the end of one row along the timeline T to cause the stimulation pattern represented by the block B to repeat.

In some embodiments, graphical presentation module 14, 114, 214 updates the display of one or more blocks B, for example, on the screen. In some embodiments, graphical presentation module 14, 114, 214 generates an updated time sequence of the stimulation patterns corresponding to the updated display of the one or more blocks B. In some embodiments, generating the updated time sequence of the stimulation patterns includes updating a start time and stop time in response to changing a location of at least one block B. In some embodiments, generating the updated time sequence of the stimulation patterns includes updating a start time or stop time in response to changing a width of at least one block B. In some embodiments, generating the updated time sequence of the stimulation patterns includes updating an up ramping or a down ramping of a stimulation amplitude associated with a block B in response to changing an edge of the block B. In some embodiments, graphical presentation module 14, 114, 214 sends the updated time sequence of the stimulation patters to stimulation pattern programming module 12, 112, 212.

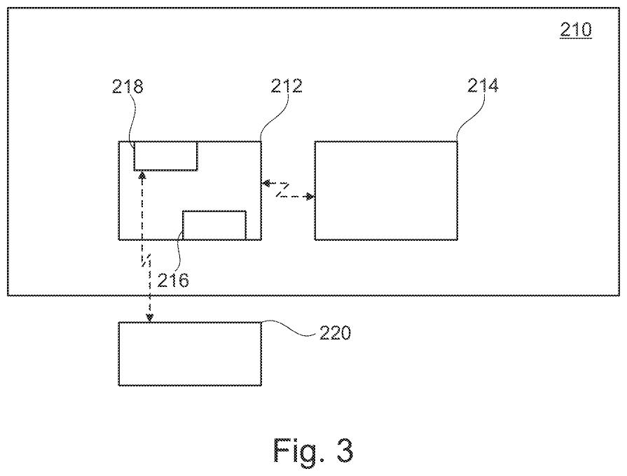

FIG. 5 depicts exemplary graphical information provided by an exemplary embodiment of a graphical presentation module 14, 114, 214, according to some embodiments of the present disclosure. In some embodiments, an interactive timeline T with blocks B that can be made larger, smaller, or moved in time (horizontally and/or on the X-axis) is shown. In some embodiments, in response to the user input, the position of a block B can be changed along one row or to a position at a different row. In some embodiments, the start and stop time, up ramping and/or down ramping, duration, position, and/or repetition and/or cycles of a block B on the timeline could be controlled and modified by a user by interacting with graphical presentation module 14, 114, 214, such as a touch screen of graphical presentation module 14, 114, 214.

In some embodiments, graphical presentation module 14, 114, 214 includes a graphical user interface, such as a display. In some embodiments, graphical presentation module 14, 114, 214 displays an illustration of at least a portion of a human body representing one or more anatomical structures. In some embodiments, the stimulation of the one or more anatomical structures evokes at least one of a motoric function or an autonomic function. For example, as shown in FIG. 5, interface module 116 displays a graphical illustration of a person, e.g. a patient walking. In some embodiments, the graphical illustration indicates what portion of a human body for which the stimulation pattern is being modulated or modified, e.g., which leg is “being programmed.”

### Creating Blocks B on a Timeline T

In some embodiments, the blocks B that are populated on the timeline T could represent stimulation patterns corresponding to muscles, muscle groups, stimulation blocks, and/or even specific muscle fibers of a patient. For example, FIG. 5 shows blocks B corresponding to “left flexion” LFLEX, “left extension” LEXT, “right flexion” RFLEX and “right extension” REXT. In some embodiments, blocks B are created based on a predefined template.

In some embodiments, blocks B are created by performing test-stimulation prior to starting the create exercises. In such a process, physiological measurements or recordings (e.g., feedback information, recordings by at least one sensor and/or camera) could be combined with test-stimulation. For example, a neuromodulation system, such as a neurostimulation system, for a patient could attempt to invoke a response from certain muscles by sending test-pulses, and a recording-system (e.g., a sensor of an external module 220) could measure the results back in. When a specific muscle, muscle group, stimulation block, specific fiber, and/or anatomical structure is detected to respond in combination with other anatomical features with similar function (for muscles this is called agonists and synergists), or otherwise if the anatomical feature responds in isolation, then the system 10, 110, 210 can distill a block B for this with a certain clinically-relevant function.

In some embodiments, system 10, 110, 210 is configured for providing test-stimulation on certain electrodes via a neuromodulation system, such as a neurostimulation system, for a patient and measure feedback information of certain muscles, such as “Iliopsoas” and “rectus femoris” on the left leg, e.g. responding strongly, via sensors, e.g. EMG sensors of an external module 220 (physiological measurements).

In some embodiments, external module 220 includes at least one camera, such as a motion-capture camera, a motion tracking video camera, or an infra-red camera. In some embodiments, the at least one camera is configured to detect or record images of a patient. In some embodiments, the at least one camera sends patient locomotion data including recorded locomotion images to stimulation pattern programming module 12, 112, 212 and/or graphical presentation module 14, 114, 214.

In some embodiments, data from the camera can be used to detect muscles that responds to a stimulation. For example, data from the camera can be used to detect muscles responsible for a locomotion (joint) action called “left hip flexion”, which is essential to walking. System 10, 110, 210 could compose a block B from this stimulation, called “left hip flexion”. This block B could represent and/or include the stimulation pattern to activate the Iliopsoas and rectus femoris on the left leg. Such a block B then could facilitate adding a time component by the timeline T. Stimulation patterns for other muscles and/or muscle groups and/or stimulation blocks and/or certain muscle fibers could be obtained similarly.

In some embodiments, blocks B could additionally and/or alternatively contain information about antagonistic muscles. For certain clinically relevant muscle stimulations, it is relevant that specific other muscles are not stimulated. A block B could also contain information about which antagonistic muscle should not be excited by its stimulation pattern.

In general, muscles could have various types of relation to a certain movement or clinical function, which should be included in the information of a block B: A muscle could be an: an agonist, an antagonist, a synergist, a neutralizer.

In some embodiments, system 10, 110, 210 could create a block B for flexing the lower arm (bending it). For example, measurements made during testing can be used to define stimulation patterns to activate the biceps. However, system 10, 110, 210 could also capture information in block B about activation of the triceps, a muscle that should not be active to achieve this flexion of the arm.

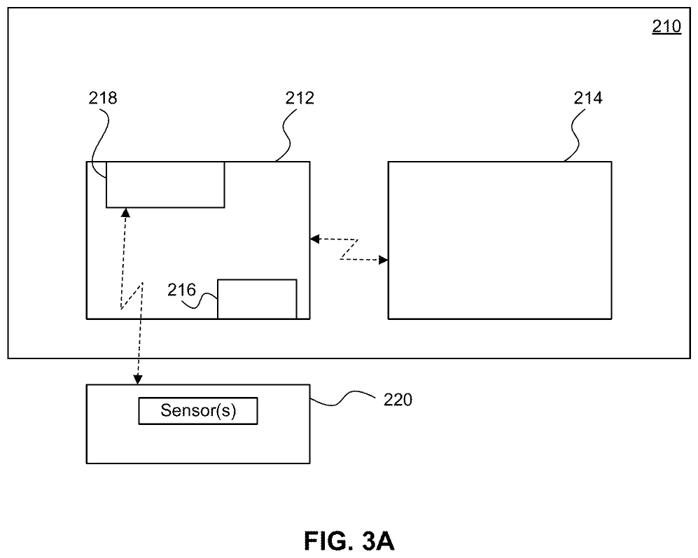

FIG. 6 depicts exemplary graphical information provided by an exemplary embodiment of a graphical presentation module 14, 114, 214, according to some embodiments of the present disclosure. FIG. 6 shows a timeline T for a “stand up” locomotion exercise. The timeline T is shown whereby left and right muscles are split up to make it more understandable.

In FIG. 6, the timeline T is split in muscular activation of the left side of the body, followed by activation of the right side of the body, to provide a more understandable and user-friendly overview. Also, an autonomic function, called bladder control, is included. In some embodiments, autonomic function can be alternatively and/or additionally provided to the exercise. In some embodiments, autonomic functions could be or could include at least one of bladder control, bowel control, sphincter control, sexual function, heart rate, blood pressure.

In some embodiments, for certain exercises, blocks B are not positioned with respect to time, but rather the progression or timeline of an exercise. For example, the progression of an exercise can be depicted as a percentage (0-100%, for e.g. closed-loop cycling, closed-loop walking), a sequence of limb positions (e.g., closed-loop cycling, closed-loop sit-to-stand), or a sequence of exercise events (e.g., gait events).

For example, a knee extension could be associated to the arbitrary time interval between a bent knee (angle between thigh and shank at 90°) and an extended knee (thigh and shank in-line).

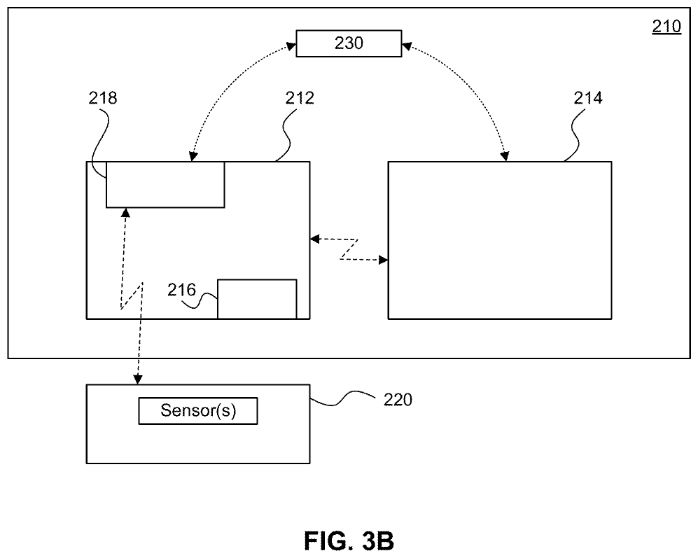

Some exercises could require a combination of the two methods (e.g., closed loop/triggered walking), where a time sequence is defined for certain exercise events (e.g., gait events). For example, a 400 ms hip flexion is associated to a detected toe-off gait event. FIG. 7 depicts exemplary graphical information provided by an exemplary embodiment of a graphical presentation module 14, 114, 214, according to some embodiments of the present disclosure.

### Block B Ramping

When blocks B are placed on the timeline T and an exercise is started for the patient, each block B can represent a stimulation pattern with a certain set of stimulation parameters: amplitude (mA/mV); pulse frequency (Hz); pulse width (uS).

In some embodiments, as soon as the block B onsets, the stimulation can be applied with these settings. When the block B ends, the stimulation can drop immediately. The clinical effect is that the patient may immediately experience the full strength of the stimulation when a block B starts. The patient may be unprepared and could be overcome by this stimulation, in particular because the stimulation may cause a violent muscular response (e.g., hip flexion). As a solution, a block B on the timeline could be modified to include ramping-up and/or ramping-down.

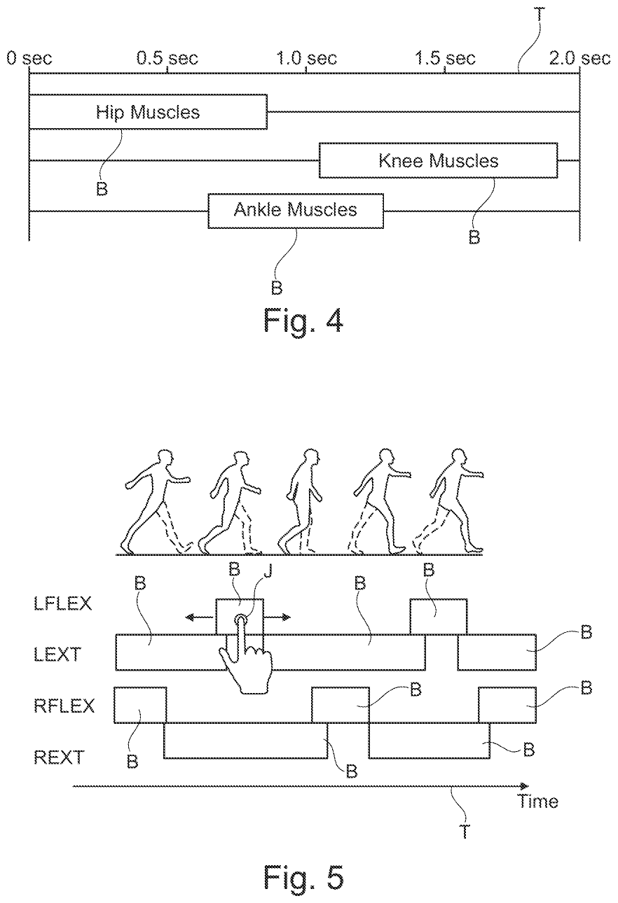

FIG. 8 depicts exemplary user interactions with graphical information displayed on a screen to ramp up or ramp down a stimulation pattern, according to some embodiments of the present disclosure. As shown in FIG. 8, in some embodiments, graphical presentation module 14, 114, 214 provides one or more handles J on block B for a user to create and manipulate up ramping and down ramping. For example, one or more handles are provided in the corners of block B. A user may interact with the handles to directly manipulate the ramping duration. In some embodiments, a user may interact with a handle on the slope of the ramp to directly manipulate the ramping angle or ramping trajectory, such as forming a non-linear curve.

Cyclic and/or Repeating Timeline T

In some embodiments, a stimulation timeline T could be automatically repeated to allow for a constant motoric function, e.g., locomotion or autonomic function, to be performed.

Locomotion can be or include, for example, walking, running, cycling, swimming. In some embodiments, the timeline T can be repeated like an “automatically replay” in these exercises. The timeline T can be cyclic and/or repeat in different modes. In some embodiments, the timeline T runs once and then stops stimulation. In some embodiments, the timeline T runs once but keep stimulating the last block indefinitely until manually stopped by the user. Such an implementation can be beneficial, for example, for standing up, where at the end stimulation should be facilitated to keep the patient standing upright. In some embodiments, the timeline T runs indefinitely until manually stopped by the user.

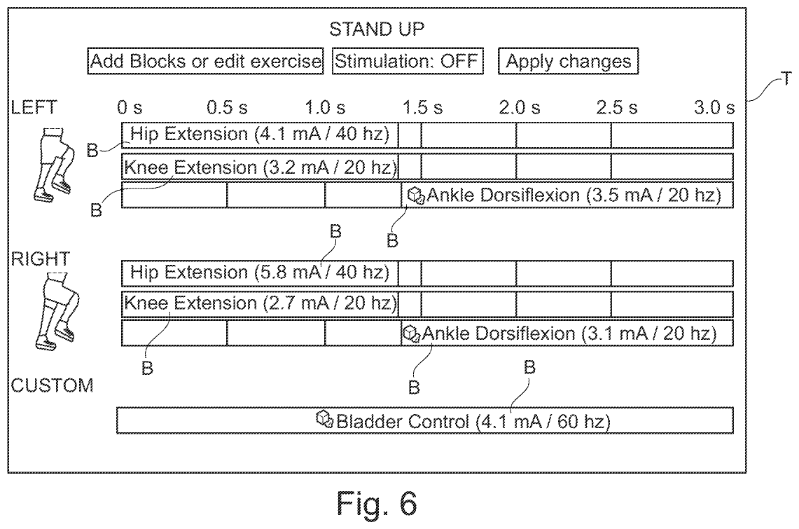

In some embodiments, a timeline T can also be embodied to be circular, to make this cyclic behavior more visual. FIG. 9 depicts an example of a timeline embodied as a circle that repeats itself, according to some embodiments of the present disclosure. As described herein, any suitable visualization method that more clearly illustrates that a timeline may be cyclic can be used for displaying the timeline, such as a circular pattern that resembles a clock.

Auditory Cues and/or Tones

To further assist a user to tune to timing (start and stop of stimulation of a muscle), in some embodiments, system 10, 110, 210 can provide acoustic information to a user or a patient concerning a start of one of the plurality of stimulation patterns or the time sequence a plurality of stimulation patterns. In some embodiments, the acoustic information includes one or more auditory tones. In some embodiments, the acoustic information starts slightly before or as soon as a block B is initiated and/or stopped.

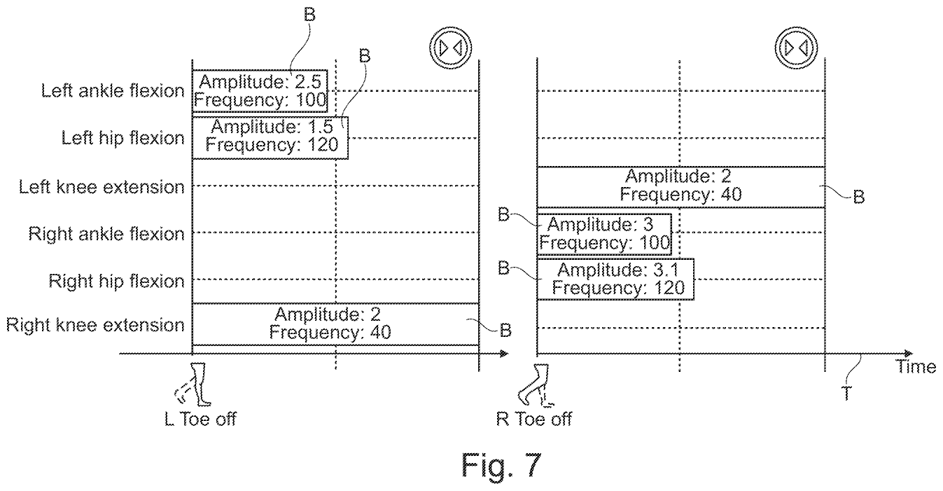

FIG. 10 depicts an example of how acoustic information can be provided, according to some embodiments of the present disclosure. In some embodiments, the acoustic information can be provided by acoustic module 230. In some embodiments, the acoustic information is provided by the neuromodulation system, such as a neurostimulation system, for a patient. In some embodiments, the acoustic information is provided by a component of the neuromodulation system, such as the controller, the programmer, the communication module, the stimulation device, the stimulation pattern programming module 12, 112, 212 or the graphical presentation module 14, 114, 214, such as a tablet. In some embodiments, the acoustic information is provided by a component used to program a pulse generator via an intermediate microprocessor.

In some embodiments, the auditory tone could be a simple tone and/or single frequency, multiple frequencies, music, and/or a vocal cue. In some embodiments, the auditory tone is provided for important muscles, muscle groups, or anatomical structures involved in the movement or function, for example, “left rectus femoris.” In some embodiments, vocal cues could provide a count-down before stimulation is applied. This could help the patient to align and prepare his self-invoked movements to align with these stimulations.

### Manual Sequencing of Blocks B

In some embodiments, the user can manually place blocks B on the timeline T (in one row or between different rows), move these blocks, and/or increase and/or decrease their durations. Alternatively, or additionally, templates can be provided for certain exercises and for patients with specific residual motor-functions. In some embodiments, templates can be provided for restoring certain autonomic functions. In some embodiments, the user can manually change the stimulation patterns and/or the time sequence of stimulation patterns or the shapes and positions of the blocks on the timeline by acting on control elements J configured for being actuated by the user. The control elements include, but not limited to anchors, axes, points, knots, buttons, arrows, hand signals, emojis, crosses, windows, text, and/or shortcuts. In general, control elements could also be referred to as joints or handles J.

Automated Sequencing of Stimulation Parameters and/or Blocks B

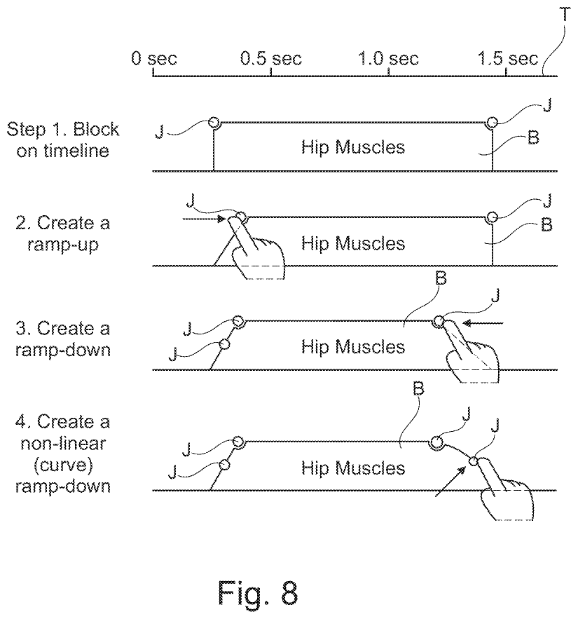

In addition to manual control, in some embodiments, other elements, such as external module 220 can be used to control or modify the contents of the timeline T. FIG. 11 depicts a schematic representation of an exemplary embodiment for updating stimulation patterns, according to some embodiments of the present disclosure. As shown in FIG. 11, external module 220 can be used to fine-tune the stimulation patterns and/or the time sequence of the stimulation patterns by using an automated process (e.g., a set of instructions/algorithms). Additionally, or alternatively, external module 220 can be used to fine-tune the stimulation patterns and/or the time sequence of the stimulation patterns by providing feedback information. In some embodiments, the automated process can be modified and set by the user. In some embodiments, one or more of the stimulation simulation patterns can be gradually increased and/or decreased at the onset or offset of a block B. In some embodiments, non-linear ramp up and/or ramp down is enabled. In some embodiments, non-linear ramp up and/or ramp down is more natural to a patient and leads to better therapy with improved clinical outcome.

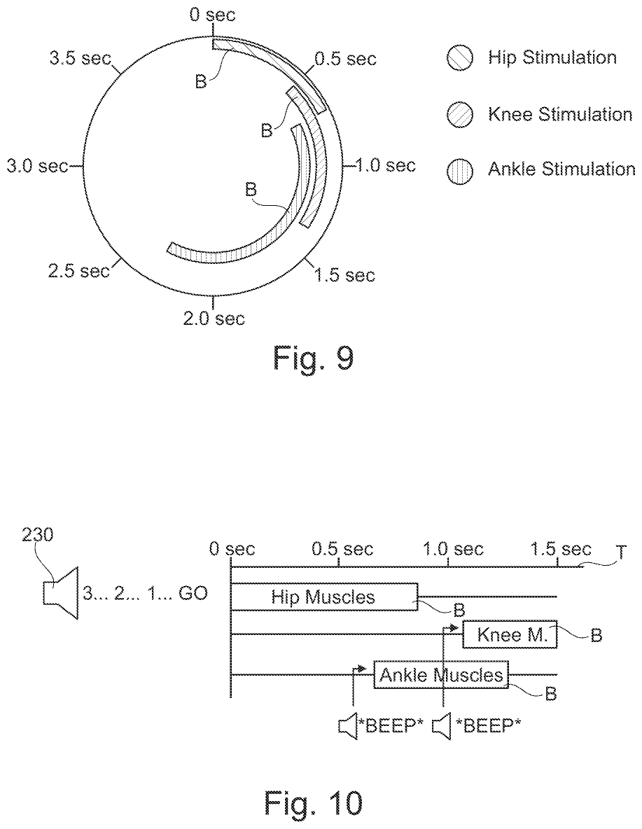

FIG. 12 depicts an example of superimposing camera recordings on intended locomotion of a patient, according to some embodiments of the present disclosure. In some embodiments, to provide the user a better guidance for improving the patient's stimulation patterns, graphical presentation module 14, 114, 214 receives patient locomotion data from a camera included by the external module 220. In some embodiments, patient locomotion data includes camera recordings, images, and/or frames of movement or locomotion of the patient. In some embodiments, graphical presentation module 14, 114, 214 display the recorded locomotion images for the patient together with reference locomotion images for the patient along the timeline. For example, graphical presentation module 14, 114, 214 may synchronize and/or superimpose the recorded locomotion images for the patient onto reference locomotion images. In some embodiments, the reference locomotion images show one or more optimal outcome patterns, such as an optimal walking pattern. In some embodiments, the optimal outcome pattern can be movements of a healthy subject or intended movements of the patient. As such, the user could see which muscles need more and/or less activation to match the optimal outcome pattern, for example, an optimal walking pattern. As described herein, the patient locomotion data can be used for restoring any suitable locomotion activity and/or motoric function.

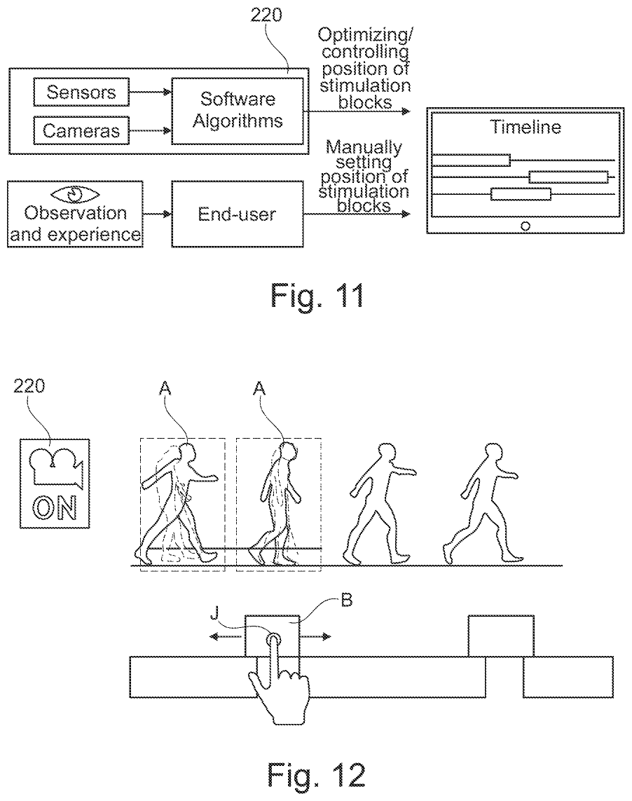

Physiological measurements, in addition to or instead of camera-recordings, can also give information on how to position or modify blocks B. In some embodiments, sensors allowing for physiological measurements could be used to better position blocks B along a timeline to align the stimulation pattern with the patient's voluntary activity. FIG. 13 depicts exemplary graphical information provided by an exemplary embodiment of a graphical presentation module 14, 114, 214, according to some embodiments of the present disclosure. In some embodiments, as shown in FIG. 13, at least one physiological measurement of the patient is added to the timeline and/or superimposed on block B to facilitate positioning of block B.

In some embodiments, one or more sensors, such as EMG sensors, may be applied on a patient to measure when muscles are activated voluntarily by the patient. In some embodiments, measurements by the one or more sensors are displayed in the same sequence, e.g., timeline T as the one or more blocks B are placed in. For example, FIG. 13 shows a timeline T with hip muscle block B and live EMG measurements of that hip. The EMG trace data could be used to better position the block B to align with the patient's voluntary activity. As such, the user has guidance to position blocks B more effectively, and to the benefit of the therapy.

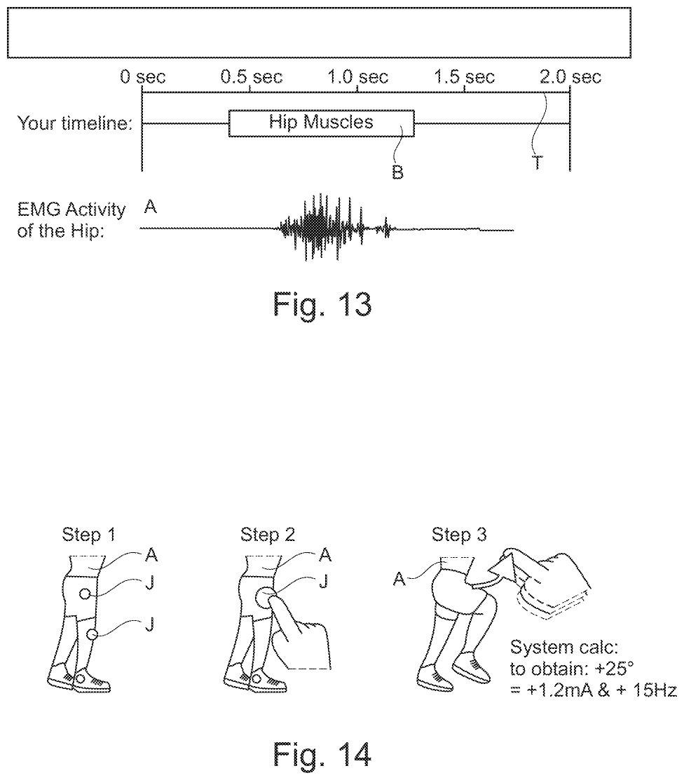

FIG. 14 depicts an exemplary of manipulation and/or modulation of movement of a patient on an exemplary avatar A, according to some embodiments of the present disclosure. In some embodiments, as shown in FIG. 14, graphical presentation module 14, 114, 214 displays a patient avatar A on a screen. The user may directly interact with an avatar A to modulate and configure the stimulation settings to obtain a certain movement or autonomic function.

In some embodiments, an avatar A can be displayed with one or more discrete joints J. As used herein, joints J can be generally referred to as elements or graphical objects that a user can interact with and use touch-based interaction to generate one or more stimulation patterns and/or a time sequence of stimulation patterns. For example, as shown in FIG. 14, in response to user input, graphical presentation module 14, 114, 214 can update a position of one or more body parts of the patient avatar A.

In some embodiments, system 10, 110, 210 can translate this change of position of one or more body parts of the patient avatar A to one or more stimulation patterns and/or a time sequence of stimulation patterns. For example, graphical presentation module 14, 114, 214 can generate one or more stimulation patterns and a tine sequence of the stimulation patterns based on the updated position of the one or more body parts of the patient avatar A. In some embodiments, stimulation pattern programming module 12, 112, 212 receives the one or more stimulation patterns and time sequence of the stimulation patterns time sequence of the plurality of stimulation patterns. In some embodiments, stimulation pattern programming module 12, 112, 212, provides, instructions to simulate to a stimulation device, using one or more electrodes, at least one muscle, at least one stimulation block, and/or at least one anatomical structure based on the stimulation patterns and/or the time sequence of the stimulation patterns. The stimulation, for example, may increase or decrease a motion, or evoke the at least one of a motoric function or an autonomic function.

For example, as shown in FIG. 14, lifting the knee higher on the avatar, can cause a stimulation pattern corresponding to this motion to increase in amplitude and/or frequency. In some embodiments, the amplitude and frequency of a stimulation pattern define the intensity of the stimulation pattern. In some embodiments, the moment in time during which the interaction occurs will determine the updated time sequence. For example, lifting the knee later in the gait cycle will cause the corresponding stimulation pattern to activate later in time.

Embodiments of the present disclosure may be embodied as a device, a system, a method, a process, a computer program product, etc. Accordingly, embodiments of the present disclosure may take the form of an entirely hardware embodiment, an entirely software embodiment, or an embodiment combining software and hardware for allowing a specialized device having the described specialized components to perform the functions described above.

Furthermore, embodiments of the present disclosure may take the form of a computer program product embodied in one or more computer-readable storage media that may be used for storing computer-readable program codes. Based on such an understanding, the technical solutions of the present disclosure can be implemented in a form of a software product. The software product can be stored in a non-volatile storage medium (which can be a CD-ROM, a USB flash memory, a mobile hard disk, and the like). The storage medium can include a set of instructions for instructing a computer device (which may be a personal computer, a server, a network device, a mobile device, or the like) or a processor to perform a part of the steps of the methods provided in the embodiments of the present disclosure.

The foregoing descriptions have been presented for purposes of illustration. They are not exhaustive and are not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

It should be noted that, the relational terms herein such as “first” and “second” are used only to differentiate an entity or operation from another entity or operation, and do not require or imply any actual relationship or sequence between these entities or operations. Moreover, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the terms “and/or” and “or” encompass all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

It is appreciated that the above-described embodiments can be implemented by hardware, or software (program codes), or a combination of hardware and software. If implemented by software, it may be stored in the above-described computer-readable media. The software, when executed by the processor can perform the disclosed methods. The computing units and other functional units described in this disclosure can be implemented by hardware, or software, or a combination of hardware and software. One of ordinary skill in the art will also understand that multiple ones of the above-described modules/units may be combined as one module/unit, and each of the above-described modules/units may be further divided into a plurality of sub-modules/sub-units.

In the foregoing specification, embodiments have been described with reference to numerous specific details that can vary from implementation to implementation. Certain adaptations and modifications of the described embodiments can be made. Other embodiments can be apparent to those skilled in the art from consideration of the specification and practice of the invention disclosed herein. It is intended that the specification and examples be considered as exemplary only, with a true scope and spirit of the invention being indicated by the following claims. It is also intended that the sequence of steps shown in figures are only for illustrative purposes and are not intended to be limited to any particular sequence of steps. As such, those skilled in the art can appreciate that these steps can be performed in a different order while implementing the same method.

What is claimed is:

1. A neuromodulation system comprising:
a user interface device configured to:
display graphical information on a screen, the graphical information comprising a plurality of graphical objects representing a time sequence of a plurality of stimulation patterns along a timeline, each of the plurality of stimulation patterns corresponding to at least one of a motoric function or an autonomic function;
change a shape or a position of at least one of the plurality of graphical objects along the timeline in response to a user input;
update the display of the plurality of graphical objects on the screen; and
generate a first updated time sequence of the stimulation patterns corresponding to the updated display of the plurality of graphical objects; and
a stimulation pattern programming device configured to:
receive the first updated time sequence of the stimulation patterns; and
provide, to a stimulation device, instructions to stimulate, using a first plurality of electrodes, one or more anatomical structures based on the first updated time sequence of the stimulation patterns.

2. The system of claim 1, wherein the user interface device is further configured to:
display an illustration of at least a portion of a human body representing the one or more anatomical structures, the stimulation of the one or more anatomical structures evoking the at least one of a motoric function or an autonomic function.

3. The system of claim 1, wherein the stimulation pattern programming device is further configured to:
receive feedback information from at least one sensor, the feedback information comprising at least one of a position, motion, or physiological measurement of a patient.

4. The system of claim 3, wherein the stimulation pattern programming device is further configured to
generate a second updated time sequence of the stimulation patterns based on at least in part on the feedback information; and
provide, to the stimulation device, instructions to stimulate, using a second plurality of electrodes, the one or more anatomical structures based on the second updated time sequence of the stimulation patterns.

5. The system of claim 1, wherein the user interface device is further configured to receive patient locomotion data from a camera.

6. The system of claim 5, wherein:
the patient locomotion data comprises recorded locomotion images for a patient; and
the user interface device is further configured to:
display the recorded locomotion images for the patient together with reference locomotion images for the patient along the timeline.

7. The system of claim 1, wherein:
changing the shape or the position of the at least one of the plurality of graphical objects comprises:
changing at least one of a width, location, or edge of at least one of the plurality of graphical objects; and
generating the first updated time sequence of the stimulation patterns comprises:
updating a start time and stop time in response to changing a location of the at least one of the plurality of graphical objects;
updating a start time or stop time in response to changing a width of the at least one of the plurality of graphical objects; or
updating an up ramping or a down ramping of a stimulation amplitude associated with the at least one of the plurality of graphical objects in response to changing an edge of the at least one of the plurality of graphical objects.

8. The system of claim 1, further comprising an acoustic device operatively connected to the user interface device and configured to provide acoustic information to a user or a patient concerning a start of:
one of the plurality of stimulation patterns; or
the time sequence.

9. The system of claim 1, wherein the user interface device configured to:
display the plurality of graphical objects along the timeline in a plurality of rows; and
change, in response to the user input, the position of at least one of the plurality of graphical objects along one row or to a position at a different row.

10. The system of claim 9, wherein the user interface device is further configured to:
change the position of at least one of the plurality of graphical objects to the end of one row along the timeline to cause the stimulation pattern represented by the at least one of the plurality of graphical objects to repeat.

11. A non-transitory computer-readable storage medium storing one or more programs that, when executed by one or more processors, cause the one or more processors to perform a method for neuromodulation, the method comprising:
displaying, by a user interface device, graphical information on a screen, the graphical information comprising a plurality of graphical objects representing a time sequence of a plurality of stimulation patterns along a timeline, each of the plurality of stimulation patterns corresponding to at least one of a motoric function or an autonomic function;
changing, by the user interface device, a shape or a position of at least one of the plurality of graphical objects along the timeline in response to a user input;
updating, by the user interface device, the display of the plurality of graphical objects on the screen;
generating, by the user interface device, a first updated time sequence of the stimulation patterns corresponding to the updated display of the plurality of graphical objects;
receiving, by a stimulation pattern programming device, the first updated time sequence of the stimulation patterns; and
providing, by the stimulation pattern programming device to a stimulation device, instructions to stimulate, using a first plurality of electrodes, one or more anatomical structures based on the first updated time sequence of the stimulation patterns.

12. The medium of claim 11, wherein the set of instructions, when executed by the one or more processors, cause the one or more processors to further perform:
displaying, by the user interface device, an illustration of at least a portion of a human body representing the one or more anatomical structures, the stimulation of the one or more anatomical structures evoking the at least one of a motoric function or an autonomic function.

13. The medium of claim 11, wherein the set of instructions, when executed by the one or more processors, cause the one or more processors to further perform:
receiving, by the stimulation pattern programming device, feedback information from at least one sensor, the feedback information comprising at least one of a position, motion, or physiological measurement of a patient.

14. The medium of claim 13, wherein the set of instructions, when executed by the one or more processors, cause the one or more processors to further perform:
generating, by the stimulation pattern programming device, a second updated time sequence of the stimulation patterns based on at least in part on the feedback information; and
providing, by the stimulation pattern programming device to the stimulation device, instructions to stimulate, using a second plurality of electrodes, the one or more anatomical structures based on the second updated time sequence of the stimulation patterns.

15. The medium of claim 11, wherein the set of instructions, when executed by the one or more processors, cause the one or more processors to further perform:
receiving, by the user interface device, patient locomotion data from a camera.

16. The medium of claim 15, wherein:
the patient locomotion data. comprises recorded locomotion images for a patient; and
the set of instructions, when executed by the one or more processors, cause the one or more processors to further perform:
displaying, by the user interface device, the recorded locomotion images for the patient together with reference locomotion images for the patient along the timeline.

17. The medium of claim 11, wherein:
changing the shape or the position of the at least one of the plurality of graphical objects comprises:
changing at least one of a width, location, or edge of at least one of the plurality of graphical objects; and
generating the first updated time sequence of the stimulation patterns comprises:
updating a start time and stop time in response to changing a location of the at least one of the plurality of graphical objects;
updating a start time or stop time in response to changing a width of the at least one of the plurality of graphical objects; or
updating an up ramping or a down ramping of a stimulation amplitude associated with the at least one of the plurality of graphical objects in response to changing an edge of the at least one of the plurality of graphical objects.

18. The medium of claim 11, wherein the set of instructions, when executed by the one or more processors, cause the one or more processors to further perform:
providing, by an acoustic device operatively connected to the user interface device, to a user or a patient concerning a start of:
one of the plurality of stimulation patterns; or
the time sequence.

19. The medium of claim 11, wherein the set of instructions, when executed by the one or more processors, cause the one or more processors to further perform:
displaying, by the user interface device, the plurality of graphical objects along the timeline in a plurality of rows; and
changing, by the user interface device, in response to the user input, the position of at least one of the plurality of graphical objects along one row or to a position at a different row.

20. The medium of claim 11, wherein the set of instructions, when executed by the one or more processors, cause the one or more processors to further perform:
changing, by the user interface device, the position of at least one of the plurality of graphical objects to the end of one row along the timeline to cause the stimulation pattern represented by the at least one of the plurality of graphical objects to repeat.

21. A neuromodulation system comprising:
a user interface device configured to:
display a patient avatar on a screen;
update a position of one or more body parts of the patient avatar in response to user input; and
generate a time sequence of a plurality of stimulation patterns based on the updated position of the one or more body parts; and
a stimulation pattern programming device configured to:
receive the time sequence of the plurality of stimulation patterns; and
provide, to a stimulation device, instructions to stimulate, using a plurality of electrodes, one or more anatomical structures of a patient based on the time sequence of the plurality of stimulation patterns.
